(19) United States
(12) Patent Application Publication
(10) Pub. No.: US 2026/0076727 A1
(43) Pub. Date: Mar. 19, 2026

(54) SPLIT-TYPE ABLATION NEEDLE AND CRYOABLATION PUNCTURE SYSTEM

(71) Applicant: SHANGHAI MAAGI MEDICAL TECHNOLOGY CO., LTD, Shanghai (CN)

(72) Inventors: Kangwei ZHANG, Shanghai (CN); Aili ZHANG, Shanghai (CN); Xingchen WAN, Shanghai (CN)

(21) Appl. No.: 19/108,989

(22) PCT Filed: Nov. 16, 2022
(86) PCT No.: PCT/CN2022/132349

§ 371 Date: Mar. 5, 2025

(30) Foreign Application Priority Data
Jun. 27, 2022 (CN) 202210737598.7

(51) Int. Cl. A61B 18/02; A61B 18/00; A61B 18/14

(52) U.S. Cl. CPC A61B 18/02; A61B 18/1477; A61B 2018/00083; A61B 2018/00172; A61B 2018/00577; A61B 2018/0262; A61B 2018/0293

(57) ABSTRACT

A split-type ablation needle connected to a cryoablation device comprising a needle arranged at a distal end of the split-type ablation needle; a tube body connected to the needle, wherein two second channels used for delivering a medium to the needle and delivering the medium returned from the needle respectively, and an insulation structure circumferentially wrapping the exterior of the two second channels, are provided inside the tube body; and a second-type joint, comprising a second connector and a second cavity. The split-type ablation needle is enable to be disassembled from the cryoablation device for puncture only, thereby reducing the impact of the tail weight of the split-type ablation needle on the puncture process.

The present application claims priority to Chinese Patent Application No. 202210737598.7, filed with the China National Intellectual Property Administration on Jun. 27, 2022 and entitled “SPLIT-TYPE ABLATION NEEDLE AND CRYOABLATION PUNCTURE SYSTEM”, which is incorporated herein by reference in its entirety.

### TECHNICAL FIELD

Embodiments of the present application relate to the technical field of medical instruments, in particular to a split-type ablation needle and a cryoablation puncture system including the split-type ablation needle.

### BACKGROUND

With the continuous development and maturity of medical imaging technologies such as magnetic resonance imaging and ultrasound imaging, minimally invasive surgeries such as low-temperature cryotherapy and thermal ablation therapy for tumors have been developed rapidly.

Multi-modal thermophysical therapy with precise control on liquid nitrogen freezing and radio frequency heating process can not only overcome the shortcomings of single cold and single heat therapy, but also can improve the cure rate of tumors and protect normal tissues from damage. Therefore, it has become a hot research direction in thermophysical therapy for tumors.

There are two main working principles of cryotherapy in the multi-modal therapy. One is based on the gas throttling effect, namely, the Joule Thomson principle. For this type of cryogenic probe that delivers high-pressure and normal temperature gas, the insulation characteristic of a tube is usually not considered, but the pressure resistance characteristics of the tube and a joint are considered. The other one is to deliver a low-temperature refrigerant such as liquid nitrogen to a needle portion and take tissue heat away through the principle of phase-change heat transfer. For this type of probe that delivers a low-temperature medium, the insulation characteristic of a tube is considered to prevent significant heat leakage, slow cooling rate, and adverse phenomena such as frosting and condensation on the outer surface of the tube.

The existing cryoablation needle usually has a one-piece structure, including a needle portion and a needle tail tube portion, the needle portion is used for directly acting on the human body, and the needle tail tube portion is used for being connected to a host device for cryoablation surgery. Especially for a cryoprobe based on the principle of phase-change heat transfer, in order to increase the insulation of a tube, the needle tail tube portion is usually designed in a vacuum insulation form. The vacuum connection tube, which is thick and heavy with poor bending and shaping performance, is not convenient for puncture by a doctor when combined with the needle, resulting in large stress. After the probe punctures in place, the weight of the needle tail tube leads to position offset of the probe, and a support device is required for assistance, making the surgical process cumbersome.

Therefore, a split-type ablation needle that can reduce the stress involved in doctor's puncture is urgently needed.

### SUMMARY

In view of the above problems, the present application provides a split-type ablation needle and a cryoablation puncture system including the split-type ablation needle to overcome or at least partially solve the above problems.

An embodiment of the present application provides a split-type ablation needle, connected to a cryoablation device, the cryoablation device being provided with a first-type joint, the first-type joint having a first connector and a first cavity, two first channels being provided in the first cavity, the two first channels being in communication with the interior of the cryoablation device for outputting and inputting a medium inside the cryoablation device respectively, where the split-type ablation needle includes: a needle arranged at a distal end of the split-type ablation needle; a tube body connected to the needle, where two second channels used for delivering the medium to the needle and delivering the medium returned from the needle respectively, and an insulation structure circumferentially wrapping the exterior of the two second channels, are provided inside the tube body; and a second-type joint, including a second connector and a second cavity, where two third channels are provided in the second cavity, and the two third channels are in communication with the two second channels respectively; the second connector is matched with the first connector, and the second connector can be assembled with or disassembled from the first connector; when the second connector is assembled with the first connector, the second-type joint and the first-type joint form a sealing structure, and the two third channels are in communication with the two first channels respectively.

Optionally, the two second channels include: a first air inlet channel used for delivering the medium to the needle; a first air return channel sleeved outside the first air inlet channel for delivering the medium returned from the needle; a second air inlet channel in communication with the first air inlet channel; and a second air return channel in communication with the first air return channel; where the second air inlet channel and the second air return channel pass through the interior of the tube body in parallel and are both made of flexible materials.

Optionally, the insulation structure includes: a seal assembly arranged between an outer wall of the first air return channel and an inner wall of the tube body, and forming an insulation zone with a proximal end of each of the first air inlet channel and the first air return channel; and a first flexible casing sleeved outside the second air inlet channel and the second air return channel, where a first insulation layer is formed between the first flexible casing and each of the second air inlet channel and the second air return channel; where the tube body is provided with a flexible portion, and the flexible portion is sleeved outside the first flexible casing to form a second insulation layer with the first flexible casing.

Optionally, the cryoablation device is further provided with a first docking portion and a first-type transition member, the first connector and the first-type transition member form a first tubular structure, and the first cavity penetrates the first tubular structure; the second-type joint is further provided with a second-type transition member, the second connector and the second-type transition member form a second tubular structure, and the second cavity penetrates the second tubular structure; the first-type transition member is cooperatively connected to the first docking portion, and the flexible portion is further provided with a third connector, where the first-type joint further includes a first insulation structure externally wrapping the two first channels and arranged between the first tubular structure and the two first channels; the second-type joint further includes a second insulation structure externally wrapping the two third channels and arranged between the second tubular structure and the two third channels; and the second-type transition member is cooperatively connected to the third connector, such that the two third channels are in communication with the two second channels respectively.

Optionally, the third connector includes: a first tubular connecting portion extending radially inward from the flexible portion; and a second tubular connecting portion extending from the first tubular connecting portion in a direction parallel to an axial direction towards one end, where the second tubular connecting portion is adapted to the second-type transition member.

Optionally, the second-type transition member includes: a tubular receiving portion used for receiving the two third channels and/or the two second channels; and a tubular fitting portion connected to the tubular receiving portion, where the outer diameter of the tubular fitting portion is ≤the inner diameter of the second tubular connecting portion, such that the tubular fitting portion is inserted into the interior of the second tubular connecting portion.

Optionally, a first locking member and a second locking member are arranged circumferentially outside the second tubular connecting portion, and the first locking member and the second locking member are cooperatively connected to apply a locking force along the outer circumference of the tubular fitting portion, so as to form a tight fit between the second tubular connecting portion and the tubular fitting portion.

Optionally, the split-type ablation needle further includes a connecting tube, and the connecting tube includes: two fourth channels; a second flexible casing sleeved outside the fourth channels, where a third insulation layer is formed between the second flexible casing and the fourth channels; a third flexible casing sleeved outside the second flexible casing to form a fourth insulation layer with the second flexible casing, where a first-type transition member and a second-type transition member are provided at two ends of the third flexible casing respectively; a first-type joint; and a second-type joint; where the first-type joint and the second-type joint are connected to the first-type transition member and the second-type transition member respectively, the first-type joint of the connecting tube is used for being assembled with or disassembled from the second-type joint of the split-type ablation needle, and the second-type joint of the connecting tube is used for being assembled with or disassembled from the first-type joint of the cryoablation device; when the first-type joint of the connecting tube is assembled with the second-type joint of the split-type ablation needle, the two fourth channels are in communication with the two third channels; when the second-type joint of the connecting tube is assembled with the first-type joint of the cryoablation device, the two fourth channels are in communication with the two first channels.

The first-type joint, the second-type joint, the first-type transition member, and the second-type transition member may all have standard structures, that is, the above structures used on different components may be the same.

Optionally, the first connector has a hollow cavity, the second connector has a protrusion matching the cavity, and the protrusion can be inserted into the cavity.

Optionally, the first connector and the second connector are further provided with a clamping member and a groove respectively; when the protrusion is inserted into the cavity, the first connector is clamped with the clamping member and the groove of the second connector.

Optionally, the first connector and the second connector are connected by threads.

Optionally, a tubular expansion portion is provided at the connection between the first air return channel and the second air return channel, the diameter of the tubular expansion portion is greater than that of the first air return channel, the tubular expansion portion has a hollow inner cavity, and the inner cavity is in communication with the first air return channel and the second air return channel respectively.

Optionally, the split-type ablation needle further includes a radio frequency cable connected to the needle for radio frequency therapy, and the radio frequency cable includes: a radio frequency signal transmission line; a temperature signal transmission line; an anti-radiation insulation layer circumferentially wrapping the radio frequency signal transmission line and the temperature signal transmission line; and a flexible cable circumferentially wrapping the anti-radiation insulation layer.

Optionally, the outer surface of the portion of the tube body corresponding to the insulation zone is coated with an electrical insulation material.

Optionally, the first flexible casing and the second flexible casing have outer diameters of 8-12 mm, and the flexible portion and the third flexible casing have outer diameters of 40-100 mm.

In another aspect, the present application further provides a cryoablation puncture system, the puncture system including: the aforementioned split-type ablation needle; and a cryoablation device provided with a first-type joint, which is provided with a body, two first channels arranged inside the body, and a first connector, where the two first channels are in communication with the interior of the cryoablation device for outputting and recovering a medium inside the cryoablation device respectively, and the first-type joint is used for being assembled with or disassembled from the second-type joint of the split-type ablation needle.

From the above technical solution, it can be seen that the split-type ablation needle in the embodiments of the present application can be assembled with or disassembled from the cryoablation device, so when an operator, such as a doctor, performs a puncture, the split-type ablation needle can be disassembled from the cryoablation device, and only the split-type ablation needle is used for the puncture, thereby reducing the impact of the tail weight of the split-type ablation needle on the puncture process. After the puncture is completed, the split-type ablation needle is assembled with the cryoablation device together for cryoablation therapy.

In addition, the flexible extension portion of the split-type ablation needle in the embodiments of the present application is a non-vacuum flexible insulation tube, which ensures a certain insulation effect, prevents frosting and condensation on the outer surface of the tube, achieves flexible tube design, facilitates bending and shaping, greatly reduces the mass compared to a vacuum tube, and greatly reduces the problem of position offset of the needle due to the heavy tail tube of the split-type ablation needle.

### BRIEF DESCRIPTION OF THE DRAWINGS

In order to describe the technical solutions in the embodiments of the present application or in the relevant art more clearly, the accompanying drawings that need to be used in the description of the embodiments or the relevant art will be briefly introduced below. Apparently, the accompanying drawings in the description below are merely some embodiments recited in the embodiments of the present application, and those of ordinary skill in the art may also derive other drawings according to these drawings.

FIGS. 1A-1B are respectively a perspective view and a cross-sectional view of an embodiment of a split-type ablation needle in the present application;

FIG. 1C is a schematic view of an embodiment of a cryoablation puncture system in the present application;

FIG. 2 is a cross-sectional view of an embodiment of a puncture segment of the split-type ablation needle in the present application;

FIG. 3 is a perspective view of an embodiment of a first tubular structure of the split-type ablation needle in the present application;

FIG. 4 is a perspective view of connection between the first tubular structure and a flexible extension segment of the split-type ablation needle in the present application;

FIG. 5 is a partial schematic view of the flexible extension segment of the split-type ablation needle in the present application;

FIG. 6 is an exploded view of the flexible extension segment docked with a second-type joint of the split-type ablation needle in the present application;

FIG. 7 is a cross-sectional view of an embodiment of a connecting tube of the split-type ablation needle in the present application;

FIGS. 8A and 8B are respectively a perspective view and a cross-sectional view of an embodiment of a split-type ablation needle including a connecting tube in the present application; and

FIG. 9 is a cross-sectional view of an embodiment of a split-type ablation needle provided with a radio frequency cable in the present application.

### REFERENCE NUMERALS OF ELEMENTS

10: Split-type ablation needle; 20: Cryoablation device; 30, 148: First-type joint; 31, 146: Second-type transition member; 32: First channel; 33: First connector; 34: First tubular structure; 111: Needle; 112: Tube body; 112a: Puncture segment tube body; 112a-1: Needle body portion; 112a-2: Grip portion; 112b: Flexible portion; 113-1: First air inlet channel; 114-1: First air return channel; 113-2: Second air inlet channel; 114-2: Second air return channel; 115: Seal assembly; 121: Second channel; 122: First flexible casing; 13, 149: Second-type joint; 131, 147: Second-type transition member; 132: Second connector; 133: Second tubular structure; 134: Third channel; 21: First docking portion; 126: Third connector; 35: First insulation structure; 135: Second insulation structure; 1261: First tubular connecting portion; 1262: Second tubular connecting portion; 1311: Tubular receiving portion; 1312: Tubular fitting portion; 1313: Connection portion; 127: First locking member; 128: Second locking member; 14: Connecting tube; 141: Fourth channel; 142: Second flexible casing; 143: Third flexible casing; 1321: Cavity; 331: Protrusion; 332: Clamping member; 1322: Groove; 15: Tubular expansion portion; 151: Inner cavity; 152: Connecting tube; 116: Radio frequency cable.

### DETAILED DESCRIPTION OF EMBODIMENTS

To make those skilled in the art better understand the technical solutions in the embodiments of the present application, the following clearly and completely describes the technical solutions in the embodiments of the present application with reference to the accompanying drawings therein. Apparently, the described embodiments are merely some but not all of the embodiments of this disclosure. All other embodiments obtained by those of ordinary skill in the art based on the embodiments of the present application should fall within the scope of protection of the embodiments of the present application.

The term “connected” includes direct connection and indirect connection. The term “communication”includes direct communication and indirect communication.

The term “proximal end” refers to an end close to the human body when the operator uses the split-type ablation needle to puncture the human body. The term “distal end” refers to an end far from the human body when the operator uses the split-type ablation needle to puncture the human body.

The term “cooperatively connected” includes mutual nesting, clamping, or locking between two structures, or any other suitable way of fixedly connecting two structures.

The following further describes specific implementations of the embodiments of the present application with reference to the accompanying drawings.

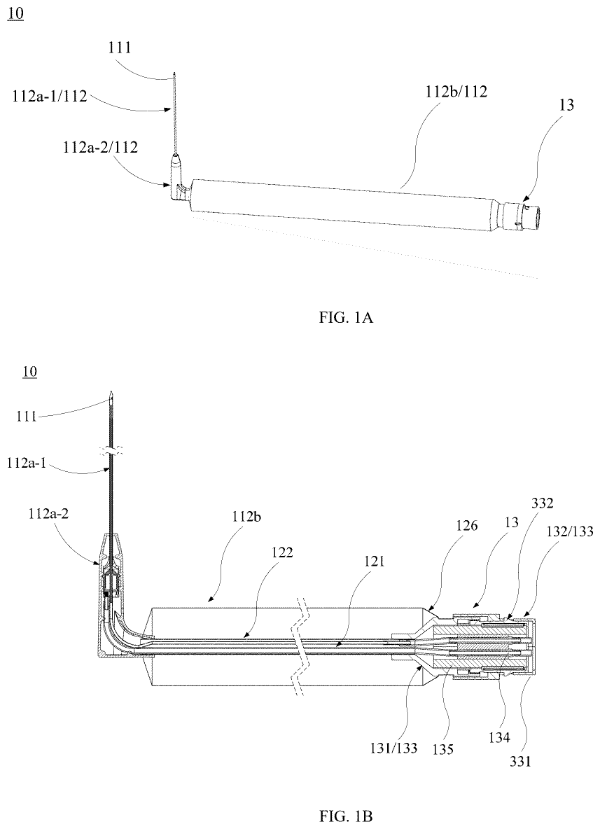

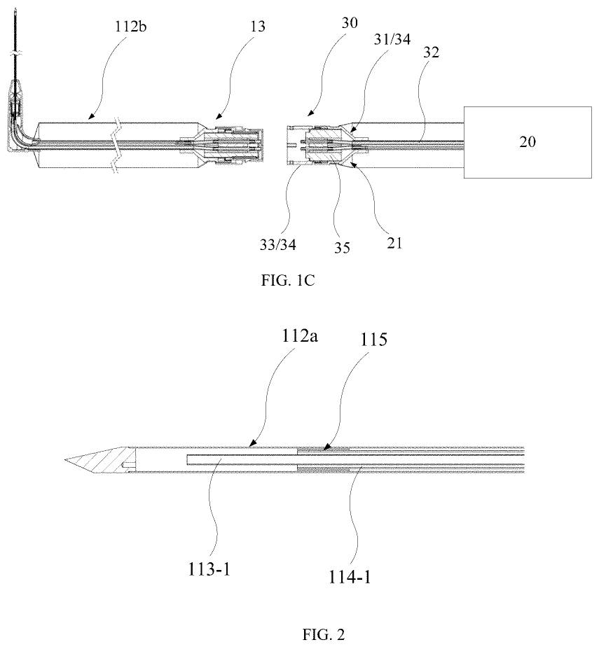

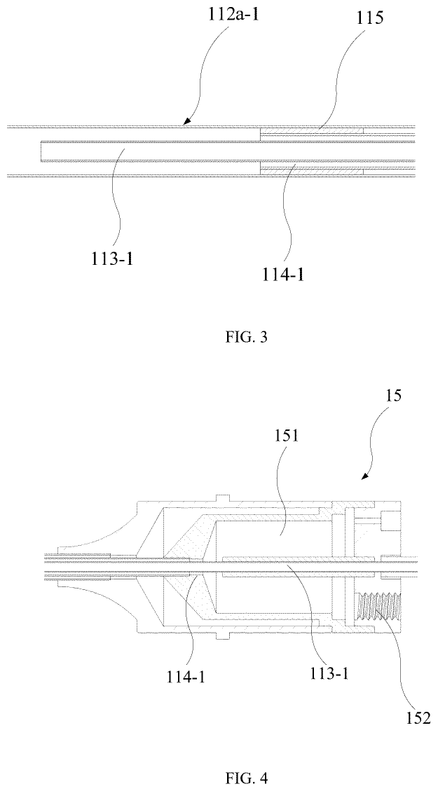

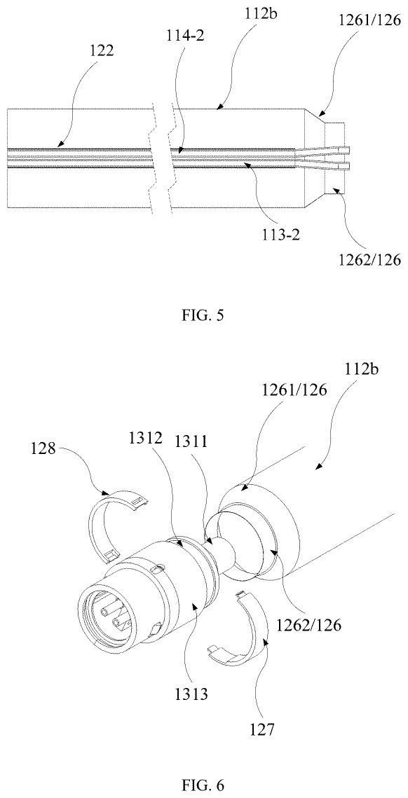

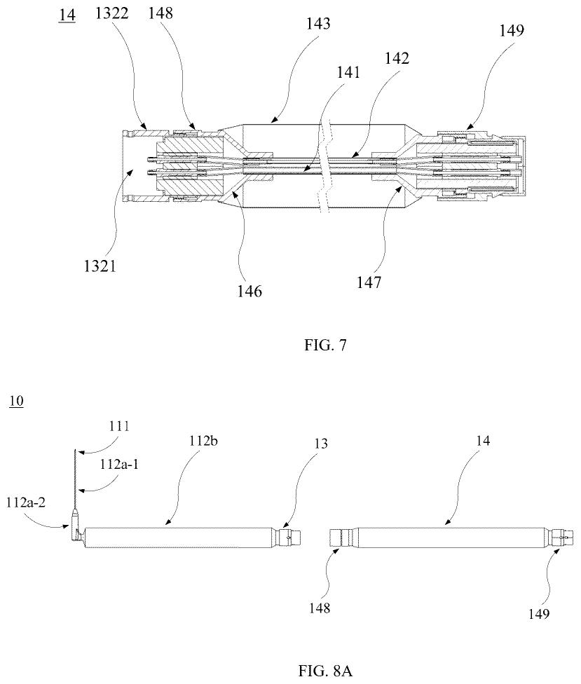

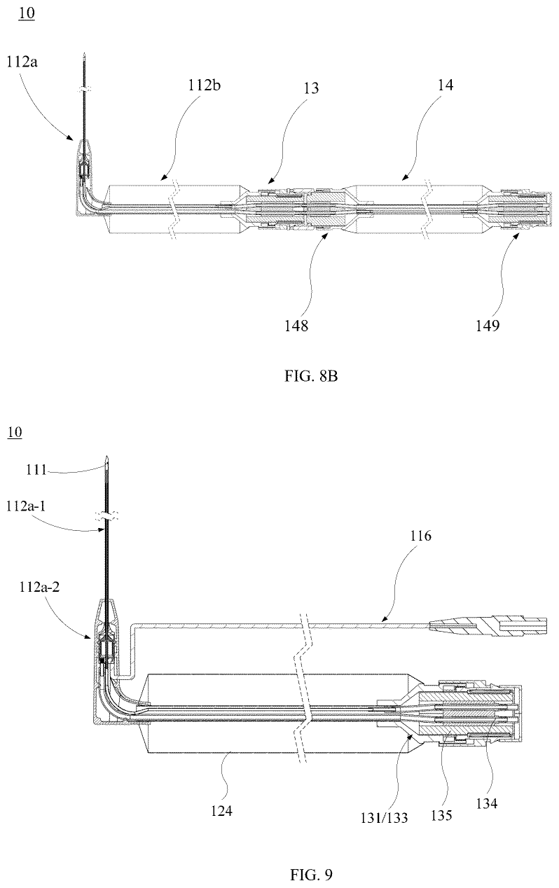

With reference to FIG. 1A to FIG. 8, in a specific implementation of the present application, a split-type ablation needle 10 is provided, connected to a cryoablation device 20. The cryoablation device 20 is provided with a first-type joint 30, the first-type joint 30 has a first connector 33 and a first cavity, and two first channels 32 are provided in the first cavity. In another embodiment, the first-type joint 30 includes a first docking portion, a first-type transition member 31, two first channels 32, and a first connector 33. The first-type transition member 31 is connected to the first connector 33 to form a first tubular structure 34, the two first channels 32 are arranged inside the first tubular structure 34, and the two first channels 32 are in communication with the interior of the cryoablation device 20 for outputting and inputting a medium inside the cryoablation device 20 respectively.

The split-type ablation needle 10 includes: a needle 111 arranged at a distal end of the split-type ablation needle 10; a tube body 112 connected to the needle, where two second channels 121 used for delivering the medium to the needle and delivering the medium returned from the needle respectively, and an insulation structure circumferentially wrapping the exterior of the two second channels 121, are provided inside the tube body; and a second-type joint 13 having a second connector 132 and a second cavity, where two third channels 134 are provided in the second cavity, and the two third channels 134 are in communication with the two second channels 121 respectively. In another embodiment, the second-type joint 13 includes: a second-type transition member 131; a second connector 132 forming a second tubular structure 133 with the second-type transition member 131; and two third channels 134 arranged inside the second tubular structure 133 and in communication with the two second channels 121 respectively. The second connector 132 is matched with the first connector 33, and the second connector 132 can be assembled with or disassembled from the first connector 33. When the second connector 132 is assembled with the first connector 33, the second-type joint 13 and the first-type joint 30 form a sealing structure, and the two third channels 134 are in communication with the two first channels 32 respectively. The tube body 112 may be provided with a puncture segment tube body 112a and a flexible portion 112b. The puncture segment tube body 112a may have a higher hardness than the flexible portion 112b to facilitate puncture operation. The puncture segment tube body 112a may further include a needle body portion 112a-1 and a grip portion 112a-2, where the needle body portion 112a-1 is connected to the needle 111, and the grip portion 112a-2 facilitates operator's grip for puncture operation. During the puncture operation, the needle 111 or the needle 111 and a portion of the needle body portion 112a-1 can puncture the human body as needed.

In one embodiment, the two second channels include a puncture segment channel and a flexible segment channel, the puncture segment channel passes through the puncture segment tube body 112a, and the flexible segment channel passes through the flexible portion 112b: the puncture segment channel includes a first air inlet channel 113-1 and a first air return channel, where the first air inlet channel 113-1 is used for delivering the medium to the needle; the first air return channel 114-1 is sleeved outside the first air inlet channel 113-1 for delivering the medium returned from the needle; the flexible segment channel includes a second air inlet channel and a second air return channel, where the second air inlet channel is in communication with the first air inlet channel 113-1; the second air return channel is in communication with the first air return channel 114-1; the second air inlet channel and the second air return channel pass through the interior of the tube body 112 in parallel and are both made of flexible materials.

In one embodiment, the insulation structure may include a puncture segment insulation structure and a flexible segment insulation structure, the puncture segment insulation structure is arranged at the puncture segment tube body 112a, and the flexible segment insulation structure is arranged at the flexible portion 112b: the puncture segment insulation structure includes a seal assembly, where the seal assembly is arranged between an outer wall of the first air return channel 114-1 and an inner wall of the tube body 112, and forms an insulation zone with a proximal end of each of the first air inlet channel 113-1 and the first air return channel 114-1; the flexible segment insulation structure includes a first flexible casing and the flexible portion of the tube body 112: the first flexible casing is sleeved outside the second air inlet channel and the second air return channel, and a first insulation layer is formed between the first flexible casing and each of the second air inlet channel and the second air return channel; the flexible portion is sleeved outside the first flexible casing to form a second insulation layer with the first flexible casing.

The split-type ablation needle 10 in the embodiments of the present application can be assembled with or disassembled from the cryoablation device 20, so when an operator, such as a doctor, performs a puncture, the split-type ablation needle 10 can be disassembled from the cryoablation device 20, and only the split-type ablation needle 10 is used for the puncture, thereby reducing the impact of the tail weight of the split-type ablation needle 10 on the puncture process. After the puncture is completed, the split-type ablation needle 10 is assembled with the cryoablation device 20 together for cryoablation therapy.

In addition, the flexible portion 112b of the split-type ablation needle 10 in the embodiments of the present application is a non-vacuum flexible insulation tube, which ensures a certain insulation effect, prevents frosting and condensation on the outer surface of the tube, achieves flexible tube design, facilitates bending and shaping, greatly reduces the mass compared to a vacuum tube, and greatly reduces the problem of position offset of the needle 111 due to the heavy tail tube of the split-type ablation needle 10.

In one embodiment of the present application, the cryoablation device 20 is further provided with a first docking portion 21, the flexible portion 112b is further provided with a third connector 126, and the structure of the first docking portion 21 can be symmetrical to the structure of the third connector 126. The first-type transition member 31 is cooperatively connected to the first docking portion 21, and the first-type joint 30 may further include a first insulation structure 35 externally wrapping the two first channels 32 and arranged between the first tubular structure 34 and the two first channels 32; the second-type joint 13 may further include a second insulation structure 135 externally wrapping the two third channels 134 and arranged between the second tubular structure 133 and the two third channels 134; the second-type transition member 131 is cooperatively connected to the third connector 126, such that the two third channels 134 are in communication with the two second channels 121 respectively. By designing the first insulation structure 35 and the second insulation structure 135, the first-type joint 30 and the second-type joint 13 are docked to form a sealing structure and achieve an insulation effect, thereby greatly alleviating frosting and condensation on the outer surface of the tube.

In one embodiment of the present application, the third connector 126 includes: a first tubular connecting portion 1261 extending radially inward from the flexible portion 124; and a second tubular connecting portion 1262 extending from the first tubular connecting portion 1261 in a direction parallel to an axial direction towards one end, where the second tubular connecting portion 1262 is adapted to the second-type transition member 131.

In one embodiment of the present application, the second-type transition member 131 includes: a tubular receiving portion 1311 used for receiving the two third channels 134 and/or the two second channels 121; and a tubular fitting portion 1312 connected to the tubular receiving portion 1311, where the outer diameter of the tubular fitting portion 1312 is ≤the inner diameter of the second tubular connecting portion 1262, such that the tubular fitting portion 1312 is inserted into the interior of the second tubular connecting portion 1262. The structures of the first-type transition member 31 and the second-type transition member 131 can be symmetrically designed and will not be repeated here. The above structures of the third connector 126 and the second-type transition member 131 can improve the sealing property of their connection and effectively support smooth docking of the third channels 134 and the second channels 121.

In one embodiment of the present application, a first locking member 127 and a second locking member 128 are arranged circumferentially outside the second tubular connecting portion 1262. The first locking member 127 and the second locking member 128 are cooperatively connected to apply a locking force along the outer circumference of the tubular fitting portion 1312, so as to form a tight fit between the second tubular connecting portion 1262 and the tubular fitting portion 1312.

In one embodiment of the present application, the split-type ablation needle 10 further includes a connecting tube 14, and the connecting tube 14 includes: two fourth channels 141; a second flexible casing 142 sleeved outside the fourth channels 141, where a third insulation layer is formed between the second flexible casing 142 and the fourth channels 141; a third flexible casing 143 sleeved outside the second flexible casing 142 to form a fourth insulation layer with the second flexible casing 142, where a first-type transition member 146 and a second-type transition member 147 are provided at two ends of the third flexible casing 143 respectively; a first-type joint 148; and a second-type joint 149; where the first-type joint 148 and the second-type joint 149 are connected to the first-type transition member 146 and the second-type transition member 147 respectively, the first-type joint 148 of the connecting tube 14 is used for being assembled with or disassembled from the second-type joint 149 of the split-type ablation needle 10, and the second-type joint 149 of the connecting tube 14 is used for being assembled with or disassembled from the first-type joint 148 of the cryoablation device 20; when the first-type joint 148 of the connecting tube 14 is assembled with the second-type joint 149 of the split-type ablation needle 10, the two fourth channels 141 are in communication with the two third channels 134; when the second-type joint 149 of the connecting tube 14 is assembled with the first-type joint 30 of the cryoablation device 20, the two fourth channels 141 are in communication with the two first channels 32. The connecting tube 14 is designed to increase the length of the tube portion of the split-type ablation needle 10, and can be assembled with or disassembled from the needle portion of the split-type ablation needle 10 (except the connecting tube 14). Therefore, when the cryoablation device 20 is far from a needle application site of a patient to be treated or inconvenient, the connecting tube 14 can be first connected to the cryoablation device 20, and the connecting tube 14 can be disassembled from the needle portion of the split-type ablation needle 10. After the doctor completes the puncture, the needle portion can be assembled with the connecting tube 14 as a whole. The connecting tube 14, which is a flexible tube, facilitates bending, greatly reduces the mass compared to a vacuum tube, has small relative stress, and greatly reduces the problem of position offset of the needle due to the heavy tail tube of the split-type ablation needle 10.

In one embodiment of the present application, the first connector 33 has a hollow cavity 1321, the second connector 132 has a protrusion 331 matching the cavity 1321, and the protrusion 331 can be inserted into the cavity 1321. There are various ways to fix the first connector 33 and the second connector 132. In one embodiment of the present application, the first connector 33 and the second connector 132 are further provided with a clamping member 332 and a groove 1322 respectively; when the protrusion 331 is inserted into the cavity 1321, the first connector 33 is clamped with the clamping member 332 and the groove 1322 of the second connector 132. In another embodiment, the first connector 33 and the second connector 132 are connected by threads. The first connector 33 and the second connector 132 may alternatively be fixed in any other suitable way. The second-type transition member 147 and the second connector 132 may be connected in various ways. For example, the second-type transition member 147 is provided with a connecting portion 1313, the connecting portion 1313 is provided with internal threads, the second connector 132 is provided with external threads, and the second-type transition member 147 and the second connector 132 are connected by the threads. However, the connection between the second-type transition member 147 and the second connector 132 is not limited to this, and may alternatively be any other suitable connection.

In one embodiment of the present application, a tubular expansion portion 15 is provided at the connection between the first air return channel 114-1 and the second air return channel 114-2, the diameter of the tubular expansion portion 15 is greater than that of the first air return channel 114-1, the tubular expansion portion 15 has a hollow inner cavity 151, and the inner cavity 151 is in communication with the first air return channel 114-1 and one of the two second channels 121 respectively, for example, the inner cavity 151 is in communication with one of the two second channels 121 through a connecting tube 152. The tubular expansion portion 15 can facilitate the doctor to grip the needle portion, prevent the needle portion with small diameter from slipping off during puncture, and facilitate the communication between the first air return channel 114-1 and the two second channels 121.

In one embodiment of the present application, the split-type ablation needle 10 further includes a radio frequency cable 116 connected to the needle 111 for radio frequency therapy, and the radio frequency cable 116 includes: a radio frequency signal transmission line; a temperature signal transmission line; an anti-radiation insulation layer circumferentially wrapping the radio frequency signal transmission line and the temperature signal transmission line; and a flexible cable circumferentially wrapping the anti-radiation insulation layer for protecting the anti-radiation insulation layer and the transmission lines therein.

In one embodiment of the present application, the outer surface of the portion of the tube body 112 corresponding to the insulation zone is coated with an electrical insulation material to prevent electrical leakage. Optionally, the first flexible casing 122 and the second flexible casing 142 have outer diameters of 8 to 12 mm, and the flexible portion 124 and the third flexible casing 143 have outer diameters of 40 to 100 mm. The materials of the two second channels 121 and the two fourth channels 141 may be stainless steel 304, polyurethane (PU), or other suitable materials. The materials of the first flexible casing 122, the flexible portion 124, the second flexible casing 142, and the third flexible casing 143 may be PVC, PE, or other suitable materials. The connections between the two second channels 121 and the two third channels 134, between the two third channels 134 and the two first channels 32, and between the two third channels 134 and the two fourth channels 141 may refer to Chinese Patent Application No. CN202122838478. X.

In another aspect, the present application further provides a cryoablation puncture system, the puncture system including: the aforementioned split-type ablation needle 10; and a cryoablation device 20 provided with a first-type joint 30, which is provided with a body, two first channels 32 arranged inside the body, and a first connector 33, where the two first channels 32 are in communication with the interior of the cryoablation device 20 for outputting and recovering a medium inside the cryoablation device 20 respectively, and the first-type joint 30 is used for being assembled with or disassembled from the second-type joint 13 of the split-type ablation needle 10. Its operation principle is explained in detail above and will not be repeated here.

Finally, it should be noted that the above embodiments are merely used for explaining the technical solutions of the embodiments of the present application, rather than limiting the present application. Although the present application is described in detail with reference to the above embodiments, those of ordinary skill in the art should understand that they can still make modifications to the technical solutions described in the above embodiments or equivalent replacements to some technical features therein, without departing from the spirit and scope of the technical solutions of the embodiments of the present application.

1. A split-type ablation needle, connected to a cryoablation device, the cryoablation device being provided with a first-type joint, the first-type joint having a first connector and a first cavity, two first channels being provided in the first cavity, the two first channels being in communication with the interior of the cryoablation device for outputting and inputting a medium inside the cryoablation device respectively, wherein the split-type ablation needle comprises:
a needle arranged at a distal end of the split-type ablation needle;
a tube body connected to the needle, the interior of the tube body being provided with:
two second channels used for delivering the medium to the needle and delivering the medium returned from the needle respectively, and
an insulation structure circumferentially wrapping the exterior of the two second channels; and
a second-type joint, comprising:
a second connector, and
a second cavity, wherein two third channels are provided in the second cavity, and the two third channels are in communication with the two second channels respectively; the second connector is matched with the first connector, and the second connector is enable to be assembled with or disassembled from the first connector; when the second connector is assembled with the first connector, the second-type joint and the first-type joint form a sealing structure, and the two third channels are in communication with the two first channels respectively.

2. The split-type ablation needle according to claim 1, wherein the two second channels comprise:
a first air inlet channel used for delivering the medium to the needle;
a first air return channel sleeved outside the first air inlet channel for delivering the medium returned from the needle;
a second air inlet channel in communication with the first air inlet channel; and
a second air return channel in communication with the first air return channel;
wherein the second air inlet channel and the second air return channel pass through the interior of the tube body in parallel and are both made of flexible materials.

3. The split-type ablation needle according to claim 2, wherein the insulation structure comprises:
a seal assembly arranged between an outer wall of the first air return channel and an inner wall of the tube body, and forming an insulation zone with a proximal end of each of the first air inlet channel and the first air return channel; and
a first flexible casing sleeved outside the second air inlet channel and the second air return channel, wherein a first insulation layer is formed between the first flexible casing and each of the second air inlet channel and the second air return channel;
wherein the tube body is provided with a flexible portion, and the flexible portion is sleeved outside the first flexible casing to form a second insulation layer with the first flexible casing.

4. The split-type ablation needle according to claim 3, wherein the cryoablation device is further provided with a first docking portion and a first-type transition member, the first-type transition member is cooperatively connected to the first docking portion, the first connector and the first-type transition member form a first tubular structure, and the first cavity penetrates the first tubular structure;
the second-type joint is further provided with a second-type transition member, the second connector and the second-type transition member form a second tubular structure, and the second cavity penetrates the second tubular structure;
the flexible portion is further provided with a third connector, wherein
the first-type joint further comprises a first insulation structure externally wrapping the two first channels and arranged between the first tubular structure and the two first channels;
the second-type joint further comprises a second insulation structure externally wrapping the two third channels and arranged between the second tubular structure and the two third channels; and
the second-type transition member is cooperatively connected to the third connector, such that the two third channels are in communication with the two second channels respectively.

5. The split-type ablation needle according to claim 4, wherein the third connector comprises:
a first tubular connecting portion extending radially inward from the flexible portion; and
a second tubular connecting portion extending from the first tubular connecting portion in a direction parallel to an axial direction towards one end, wherein the second tubular connecting portion is adapted to the second-type transition member.

6. The split-type ablation needle according to claim 5, wherein the second-type transition member comprises:
a tubular receiving portion used for receiving the two third channels and/or the two second channels; and
a tubular fitting portion connected to the tubular receiving portion, wherein the outer diameter of the tubular fitting portion is ≤the inner diameter of the second tubular connecting portion, such that the tubular fitting portion is inserted into the interior of the second tubular connecting portion.

7. The split-type ablation needle according to claim 6, wherein a first locking member and a second locking member are arranged circumferentially outside the second tubular connecting portion, and the first locking member and the second locking member are cooperatively connected to apply a locking force along the outer circumference of the tubular fitting portion, so as to form a tight fit between the second tubular connecting portion and the tubular fitting portion.

8. The split-type ablation needle according to claim 4, wherein the split-type ablation needle further comprises a connecting tube, and the connecting tube comprises:
two fourth channels;
a second flexible casing sleeved outside the fourth channels, wherein a third insulation layer is formed between the second flexible casing and the fourth channels;
a third flexible casing sleeved outside the second flexible casing to form a fourth insulation layer with the second flexible casing, wherein a first-type transition member and a second-type transition member are provided at two ends of the third flexible casing respectively;
a first-type joint; and
a second-type joint;
wherein the first-type joint and the second-type joint are connected to the first-type transition member and the second-type transition member respectively, the first-type joint of the connecting tube is used for being assembled with or disassembled from the second-type joint of the split-type ablation needle, and the second-type joint of the connecting tube is used for being assembled with or disassembled from the first-type joint of the cryoablation device; when the first-type joint of the connecting tube is assembled with the second-type joint of the split-type ablation needle, the two fourth channels are in communication with the two third channels; when the second-type joint of the connecting tube is assembled with the first-type joint of the cryoablation device, the two fourth channels are in communication with the two first channels.

9. The split-type ablation needle according to claim 1, wherein the first connector has a hollow cavity, the second connector has a protrusion matching the cavity, and the protrusion is enable to be inserted into the cavity.

10. The split-type ablation needle according to claim 9, wherein the first connector and the second connector are further provided with a clamping member and a groove respectively; when the protrusion is inserted into the cavity, the first connector is clamped with the clamping member and the groove of the second connector.

11. The split-type ablation needle according to claim 9, wherein the first connector and the second connector are connected by threads.

12. The split-type ablation needle according to claim 2, wherein a tubular expansion portion is provided at the connection between the first air return channel and the second air return channel, the diameter of the tubular expansion portion is greater than that of the first air return channel, the tubular expansion portion has a hollow inner cavity, and the inner cavity is in communication with the first air return channel and the second air return channel respectively.

13. The split-type ablation needle according to claim 1, wherein the split-type ablation needle further comprises a radio frequency cable connected to the needle for radio frequency therapy, and the radio frequency cable comprises:
a radio frequency signal transmission line;
a temperature signal transmission line;
an anti-radiation insulation layer circumferentially wrapping the radio frequency signal transmission line and the temperature signal transmission line; and
a flexible cable circumferentially wrapping the anti-radiation insulation layer.

14. The split-type ablation needle according to claim 1, wherein the outer surface of the portion of the tube body corresponding to the insulation zone is coated with an electrical insulation material.

15. The split-type ablation needle according to claim 8, wherein the first flexible casing and the second flexible casing have outer diameters of 8-12 mm, and the flexible portion and the third flexible casing have outer diameters of 40-100 mm.

16. A cryoablation puncture system, wherein the puncture system comprises:
the split-type ablation needle according to claim 1; and
a cryoablation device provided with a first-type joint, which is provided with a body, two first channels arranged inside the body, and a first connector, wherein the two first channels are in communication with the interior of the cryoablation device for outputting and recovering a medium inside the cryoablation device respectively, and the first-type joint is used for being assembled with or disassembled from the second-type joint of the split-type ablation needle.
